(19) United States
(12) Patent Application Publication
(10) Pub. No.: US 2026/0331227 A1
(43) Pub. Date: Oct. 22, 2026

(54) RECORDING MEDIUM, INFORMATION PROCESSING METHOD, AND INFORMATION PROCESSING DEVICE

(71) Applicant: Fujitsu Limited, Kawasaki-shi (JP)

(72) Inventor: Yasunari HIKIMA, Kawasaki (JP)

(73) Assignee: Fujitsu Limited, Kawasaki-shi (JP)

(21) Appl. No.: 19/568,596

(22) Filed: Mar. 16, 2026

(30) Foreign Application Priority Data
Mar. 18, 2025 (JP) 2025-043752

(51) Int. Cl. G16C 10/00; G06F 17/11

(52) U.S. Cl. CPC G16C 10/00; G06F 17/11

(57) ABSTRACT

A computer-readable recording medium stores therein a program for causing a computer to execute a process, the process including: calculating density matrices respectively corresponding to a plurality of fragments obtained by dividing a structure of a molecule based on a Lagrange multiplier; and updating the Lagrangian multiplier based on the calculated density matrices when a predetermined exit condition is not satisfied, the Lagrangian multiplier being updated using a noise-resistant optimization calculation method according to a Newton-Krylov method so as to maintain consistency among the plurality of fragments. The process is repeatedly executed until the predetermined exit condition is satisfied.

### CROSS REFERENCE TO RELATED APPLICATIONS

This application is based upon and claims the benefit of priority of the prior Japanese Patent Application No. 2025-043752, filed on Mar. 18, 2025, the entire contents of which are incorporated herein by reference.

### FIELD

The embodiments discussed herein are related to a computer-readable recording medium, an information processing method, and an information processing device.

### BACKGROUND

Conventionally, in the field of drug discovery, material development, or the like, there is a quantum chemical calculation method for analyzing the structure or properties of a molecule that is a candidate for a drug or a material. In the quantum chemical calculation, for example, the energy of a molecule is calculated. The energy is the ground or excitation energy. Here, in order to reduce the processing amount of the quantum chemical calculation, there is a molecule dividing method of dividing the structure of a molecule into multiple fragments and then calculating energy.

As the related art, for example, there is a molecule dividing method called Bootstrap Embedding (BE) in which a series of processes of calculating a density matrix corresponding to fragments and performing optimization calculation so as to maintain consistency between the fragments is iteratively performed. Further, for example, there is a molecule dividing method called Density Matrix Embedding Theory (DMET) in which an interaction between fragments is treated as a bath orbital. For example, refer to Ye, Hong-Zhou, et al. “Bootstrap embedding for molecules.” Journal of chemical theory and computation 15.8 (2019): 4497-4506.

### SUMMARY

According to an aspect of an embodiment, a computer-readable recording medium stores therein a program for causing a computer to execute a process, the process including: calculating density matrices respectively corresponding to a plurality of fragments obtained by dividing a structure of a molecule based on a Lagrange multiplier; and updating the Lagrangian multiplier based on the calculated density matrices when a predetermined exit condition is not satisfied, the Lagrangian multiplier being updated using a noise-resistant optimization calculation method according to a Newton-Krylov method so as to maintain consistency among the plurality of fragments. The process is repeatedly executed until the predetermined exit condition is satisfied.

The object and advantages of the disclosure will be realized and attained by means of the elements and combinations particularly pointed out in the claims.

It is to be understood that both the foregoing general description and the following detailed description are exemplary and explanatory and are not restrictive of the disclosure.

### BRIEF DESCRIPTION OF DRAWINGS

FIG. 1 is an explanatory diagram depicting an example of an information processing method according to an embodiment.

FIG. 2 is an explanatory diagram depicting an example of an information processing system 200.

FIG. 3 is a block diagram of an example of a hardware configuration of an information processing device 100.

FIG. 4 is a block diagram depicting an example of a functional configuration of the information processing device 100.

FIG. 5 is an explanatory diagram depicting an example of operation of the information processing device 100.

FIG. 6 is an explanatory diagram depicting an example of an effect of the information processing device 100.

FIGS. 7A, 7B, and 7C are each an explanatory diagram depicting an example of an effect of the information processing device 100.

FIG. 8 is a flowchart depicting an example of a procedure of an overall process.

### DESCRIPTION OF EMBODIMENTS

First, problems associated with the conventional techniques are discussed. In the related art, the accuracy of calculating the energy of the molecule using the molecule dividing method may decrease. For example, in an actual machine of a quantum computer, the accuracy of calculating the energy of a molecule using the molecule dividing method may be reduced due to noise.

Embodiments of an computer-readable recording medium, an information processing method, and an information processing device according to the present disclosure will be explained in detail below with reference to the accompanying drawings.

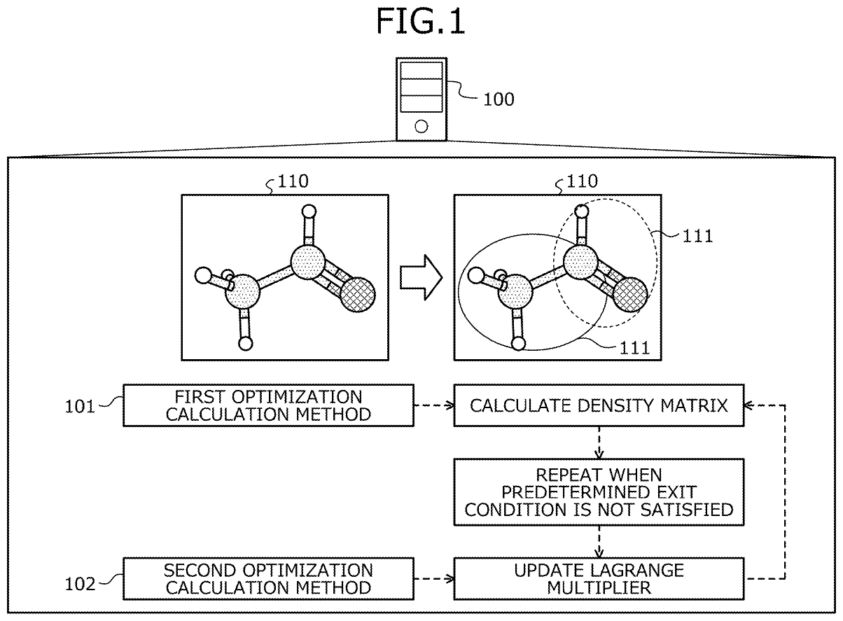

FIG. 1 is an explanatory diagram depicting an example of an information processing method according to an embodiment. The information processing device 100 is a computer for improving the accuracy of calculating the energy of a molecule. The energy is, for example, the ground energy or excitation energy of the molecule. The information processing device 100 is, for example, a server or a personal computer (PC).

Conventionally, the energy of a molecule may be calculated using a specific calculation method. The specific calculation method is, for example, coupled cluster (CC), full configuration interaction (FCI), variational quantum eigen-solver (VQE), or the like. The CC is, for example, coupled-cluster calculation with single and double excitations (CCSD).

Here, as the scale of the molecule increases, the processing amount necessary for calculating the energy of the molecule tends to increase. The scale is the number of atoms forming the molecule, etc. In order to reduce the amount of processing, there is a molecule dividing method in which the structure of a molecule is divided into multiple fragments and then energy is calculated. Examples of the molecule dividing method include BE and DMET.

BE specifically divides the structure of a molecule into multiple fragments. Specifically, BE repeats a series of processes including calculating a density matrix corresponding to each fragment and performing optimization calculation to maintain consistency between fragments, based on the density matrix when a predetermined exit condition is not satisfied, the series of processes being repeated until the predetermined exit condition is satisfied. The optimization calculation is performed by, for example, Newton's method. Regarding BE, for example, refer to Ye, Hong-Zhou, et al, “Bootstrap embedding for molecules.”

However, in the related art, the accuracy of calculating the energy of the molecule using the molecule dividing method may decrease. For example, the Newton method for performing optimization calculation in the processes of the molecule dividing method is an algorithm based on a premise that no noise is generated in the measurement of the objective function. For this reason, in a case where the energy of the molecule is calculated using the molecule dividing method in an environment in which noise may occur, the accuracy of calculating the energy of the molecule may decrease.

An environment in which noise may occur is, for example, an actual machine of a quantum computer. The noise is, for example, environmental noise, interference noise between quantum bits, noise during operation of quantum bits, or the like. An environment in which noise may occur is, for example, a quantum simulator with noise.

Therefore, in the present embodiment, an information processing method capable of improving the accuracy of calculating the energy of a molecule using a molecule dividing method will be described.

In FIG. 1, the information processing device 100 may use a first optimization calculation method 101. For example, the information processing device 100 itself may perform the first optimization calculation method 101. The first optimization calculation method 101 is, for example, CCSD, FCI, VQE, or the like. Further, the information processing device 100 may use a second optimization calculation method 102 having noise resistance. For example, the information processing device 100 itself may perform the second optimization calculation method 102. The second optimization calculation method 102 is, for example, a Newton method having noise resistance. Regarding the Newton method having noise resistance, for example, Saab, Khaled Kamal, and Samer Said Saab. “A stochastic Newton-Raphson method with noisy function measurements.” IEEE signal processing letters 23.3 (2015): 361-365 may be referred to.

The information processing device 100 obtains structural information indicating a molecular structure 110. The structural information represents, for example, the position and type of each of one or more atoms forming a molecule. The structural information represents, for example, a connection relationship between atoms. The information processing device 100 obtains the structure information by receiving the input of the structure information, based on the operation input of the user.

The information processing device 100 obtains fragment information indicating each fragment 111 among multiple fragments 111 obtained by dividing the molecular structure 110. The fragment information represents, for example, the position and the type of each of one or more atoms belonging to the fragment 111. The fragments 111 share, for example, any atom. The information processing device 100 obtains the fragment information by receiving an input of the fragment information, based on an operation input of the user, for example.

The information processing device 100 stores a predetermined exit condition. The predetermined exit condition is set in advance by the user, for example. The predetermined exit condition is, for example, that the density matrices corresponding to the respective fragments 111 match or are similar. Specifically, the information processing device 100 repeatedly executes a series of processes described in (1-1) and (1-2) below until the predetermined exit condition is satisfied.

(1-1) The information processing device 100 uses the first optimization calculation method 101 to calculate a density matrix corresponding to each fragment 111 based on a Lagrange multiplier.

(1-2) When the predetermined exit condition is not satisfied, the information processing device 100 updates the Lagrange multiplier using the second optimization calculation method 102 so as to maintain consistency among the multiple fragments 111. For example, the information processing device 100 updates the Lagrange multiplier so as to maintain the consistency between the fragments 111 according to the Newton-Krylov method, using the second optimization calculation method 102 based on the calculated density matrices. Regarding the Newton-Krylov method, for example, refer to Knoll, Dana A., and Keyes, David E. “Jacobian-free Newton-Krylov methods: A survey of approaches and applications.” Journal of Computational Physics 193.2 (2004): 357-397.

Thus, the information processing device 100 may improve the accuracy of calculating the energy of the molecule using the molecule dividing method. For example, the information processing device 100 may suppress the adverse effect of noise on the optimization calculation for updating the Lagrange multiplier in the process of calculating the energy of the molecule using the molecule dividing method. Further, the information processing device 100 may reduce the amount of processing necessary to calculate the energy of the molecule using the molecule dividing method.

(1-3) When a predetermined exit condition is satisfied, the information processing device 100 may calculate the energy of the molecule based on the Lagrange multiplier updated most recently and the density matrix calculated most recently. Thus, the information processing device 100 may appropriately calculate the energy of the molecule and complete the quantum chemical calculation.

Here, while a case in which the information processing device 100 receives the input of the structure information based on the operation input of the user has been described, the present disclosure is not limited hereto. For example, the information processing device 100 may obtain the structure information by receiving the structure information from another computer.

Here, while a case in which the information processing device 100 receives an input of fragment information has been described, the present disclosure is not limited hereto. For example, the information processing device 100 may obtain fragment information by receiving the fragment information from another computer. For example, the information processing device 100 may obtain fragment information by dividing the molecular structure 110 into multiple fragments 111 based on structure information and may generate fragment information.

Here, while a case in which functions as the information processing device 100 are realized by a single computer has been described, the present disclosure is not limited hereto. For example, functions of the information processing device 100 may be realized by cooperation of multiple computers. For example, functions of the information processing device 100 may be implemented on a cloud.

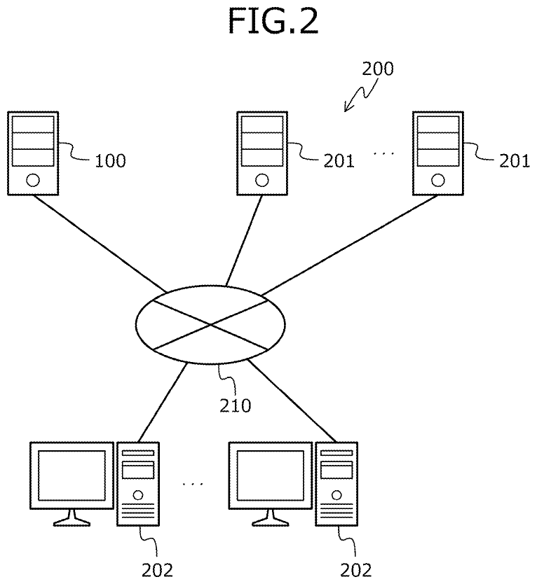

Next, an example of an information processing system 200 to which the information processing device 100 depicted in FIG. 1 is applied will be described with reference to FIG. 2.

FIG. 2 is an explanatory diagram depicting an example of the information processing system 200. In FIG. 2, the information processing system 200 includes the information processing device 100, one or more chemical calculating devices 201, and one or more client devices 202.

In the information processing system 200, the information processing device 100 and the one or more chemical calculating devices 201 are connected via a wired or wireless network 210. The network 210 is, for example, a local area network (LAN), a wide area network (WAN), the Internet, or the like. In the information processing system 200, the information processing device 100 and the one or more client devices 202 are connected via the wired or wireless network 210.

The information processing device 100 is a computer for performing a quantum chemical calculation for a molecule of interest using a molecule dividing method. Quantum chemical calculations include, for example, calculating the energy of a molecule of interest. The energy is, for example, ground energy or excitation energy. The information processing device 100 obtains a processing request requesting execution of quantum chemical calculation for a molecule of interest by using the molecule dividing method.

The processing request includes, for example, structural information indicating the structure of the molecule of interest. The structural information includes, for example, coordinates of each atom among one or more atoms forming the molecule of interest. The structural information includes, for example, the type of each of the one or more atoms forming the molecule of interest. The processing request may include, for example, a division number indicating how many fragments the structure of the molecule of interest is to be divided into. The number of divisions may be set in advance by the user, for example.

The information processing device 100 identifies the structure of the molecule of interest based on the structure information included in the processing request. The information processing device 100 generates fragment information indicating each fragment by dividing the structure of the identified molecule of interest into multiple fragments corresponding to the number of divisions. The information processing device 100 sets an initial value of the Lagrange multiplier. The information processing device 100 performs quantum chemical calculation for calculating the energy of the molecule of interest, based on the fragment information.

For example, the information processing device 100 repeatedly performs a series of processes until a predetermined exit condition is satisfied. The series of processes includes, for example, a process of calculating a density matrix corresponding to each fragment based on a Lagrange multiplier using the first optimization calculation method. The series of processes includes, for example, a process of updating the Lagrangian multiplier so as to maintain the consistency between the fragments by using the second optimization calculation method having noise resistance, when the predetermined exit condition is not satisfied.

Specifically, the information processing device 100 may communicate with a chemical calculating device 201 that performs the first optimization calculation method. Specifically, the information processing device 100 controls the chemical calculating device 201 to calculate a density matrix corresponding to each fragment, based on the Lagrange multiplier. Specifically, the information processing device 100 may communicate with a chemical calculating device 201 that performs the second optimization calculation method. Specifically, the information processing device 100 controls the chemical calculating device 201 to update the Lagrange multiplier according to the Newton-Krylov method, based on the calculated density matrices, so as to maintain consistency between fragments.

For example, when a predetermined exit condition is satisfied, the information processing device 100 calculates the energy of the molecule of interest based on the Lagrange multiplier updated most recently and the density matrix calculated most recently. Specifically, the information processing device 100 controls the chemical calculating device 201 to calculate the energy of the molecule of interest based on the Lagrange multiplier updated most recently and the density matrix calculated most recently. Specifically, the information processing device 100 receives the energy of the molecule of interest from the chemical calculating device 201.

The information processing device 100 outputs the calculated energy of the molecule of interest as a result of performing the quantum chemical calculation for the molecule of interest. The output format is, for example, display on a display, print output to a printer, transmission to another computer, or storage to a storage area. The other computer is, for example, the client device 202. The information processing device 100 may output the calculated energy of the molecule of interest as a result of performing the quantum chemical calculation for the molecule of interest, so that the user may refer to the energy. The information processing device 100 is, for example, a server or a PC.

The chemical calculating device 201 is a computer for performing quantum chemical calculations for molecules. The chemical calculating device 201 calculates a density matrix corresponding to each fragment, based on the Lagrange multiplier and using the first optimization calculation method under the control of the information processing device 100. For example, the chemical calculating device 201 may calculate for each fragment, a corresponding density matrix in cooperation with another chemical calculating device 201. The chemical calculating device 201 transmits the density matrices respectively corresponding to the fragments to the information processing device 100.

Under the control of the information processing device 100, the chemical calculating device 201 uses the second optimization calculation method and updates the Lagrange multiplier based on the density matrices so as to maintain consistency between the fragments. The update follows, for example, the Newton-Krylov method. For example, the chemical calculating device 201 may update the Lagrange multiplier in cooperation with another chemical calculating device 201. The chemical calculating device 201 transmits, for example, the Lagrange multiplier to the information processing device 100.

The chemical calculating device 201 calculates the energy of the molecule of interest based on the most recently updated Lagrange multiplier and the most recently calculated density matrix, under the control of the information processing device 100. For example, the chemical calculating device 201 may calculate the energy of the molecule of interest in cooperation with another chemical calculating device 201. The chemical calculating device 201 transmits the energy of the molecule of interest to the information processing device 100. The chemical calculating device 201 is, for example, a server or a PC. The chemical calculating device 201 may be, for example, an actual machine of a quantum computer.

The client device 202 is a computer utilized by a user who desires to perform a quantum chemical calculation for a molecule of interest. The user is, for example, an operator. In response to an operation input of the user, the client device 202 generates a processing request requesting, for the molecule of interest, execution of the quantum chemical calculation using the molecule dividing method. The client device 202 obtains structure information indicating the structure of the molecule of interest, for example, in response to an operation input by the user. The client device 202 generates, for example, a processing request including the structural information indicating the structure of the molecule of interest.

The client device 202 transmits the generated processing request to the information processing device 100. The client device 202 receives, from the information processing device 100, a result of performing the quantum chemical calculation for the molecule of interest. The client device 202 outputs the result of performing the quantum chemical calculation for the molecule of interest so that the user may refer to the result. The client device 202 is, for example, a PC, a tablet terminal, or a smartphone.

Here, while a case in which the information processing device 100 is a device different from the chemical calculating device 201 has been described, the present disclosure is not limited hereto. For example, the information processing device 100 may have a function as the chemical calculating device 201 and may also operate as the chemical calculating device 201. In this case, the information processing system 200 may omit the chemical calculating device 201.

Here, while a case in which the information processing device 100 is a device different from the client device 202 has been described, the present disclosure is not limited hereto. For example, the information processing device 100 may have a function as the client device 202 and may also operate as the client device 202. In this case, the information processing system 200 may omit the client device 202.

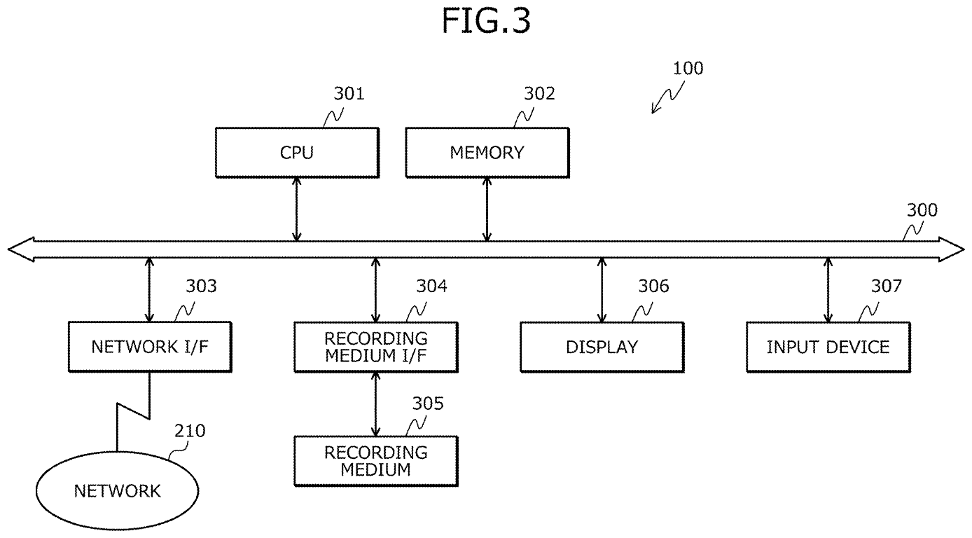

Next, with reference to FIG. 3, an example of a hardware configuration of the information processing device 100 is described.

FIG. 3 is a block diagram of an example of the hardware configuration of the information processing device 100. In FIG. 3, the information processing device 100 has a central processing unit (CPU) 301, a memory 302, and a network interface (I/F) 303. The information processing device 100 also has a recording medium I/F 304, a recording medium 305, a display 306, and an input device 307. Further, the components are connected to each other by a bus 300.

Here, the CPU 301 governs overall control of the information processing device 100. The memory 302, for example, includes a read-only memory (ROM), a random-access memory (RAM), and a flash-ROM. In particular, for example, the flash-ROM and/or ROM stores therein various programs and the RAM is used as a work area of the CPU 301. Programs stored to the memory 302 are loaded onto the CPU 301, whereby encoded processes are executed by the CPU 301.

The network I/F 303 is connected to the network 210 via a communications line and is connected to other computers through the network 210. Further, the network I/F 303 administers an internal interface with the network 210 and controls the input and output of data with respect to the other computers. The network I/F 303, for example, is a modem, a LAN adapter, or the like.

The recording medium I/F 304 controls the reading and writing of data with respect to the recording medium 305 under the control of the CPU 301. The recording medium I/F 304 is, for example, a disk drive, a solid-state drive (SSD), a universal serial bus (USB) port, or the like. The recording medium 305 is a nonvolatile memory storing data written thereto under the control of the recording medium I/F 304. The recording medium 305 is, for example, a disk, a semiconductor memory, a USB memory, or the like. The recording medium 305 may be removable from the information processing device 100.

The display 306 displays data such as a cursor, icons, toolboxes, documents, images, or functional information. The display 306 is, for example, a cathode ray tube (CRT), a liquid crystal display, or an organic electroluminescence (EL) display. The input device 307 includes keys for inputting characters, numbers, or various instructions, and inputs data. The input device 307 is, for example, a keyboard or a mouse. The input device 307 may be, for example, a touch panel-type input pad, a numeric keypad, or the like.

The information processing device 100 may include, for example, a camera in addition to the above-described components. Further, the information processing device 100 may include, for example, a printer, a scanner, a microphone, a speaker, or the like in addition to the above-described components. The information processing device 100 may include, for example, the recording medium I/F 304 and/or the recording medium 305 in plural. The information processing device 100 may omit, for example, the display 306 and/or the input device 307. The information processing device 100 may omit the recording medium I/F 304 and the recording medium 305, for example.

An example of a hardware configuration of the chemical calculating device 201 is similar to the example of the hardware configuration of the information processing device 100 depicted in FIG. 3 and thus, description thereof will be omitted.

An example of a hardware configuration of the client device 202 is similar to the example of the hardware configuration of the information processing device 100 depicted in FIG. 3 and thus, description thereof will be omitted.

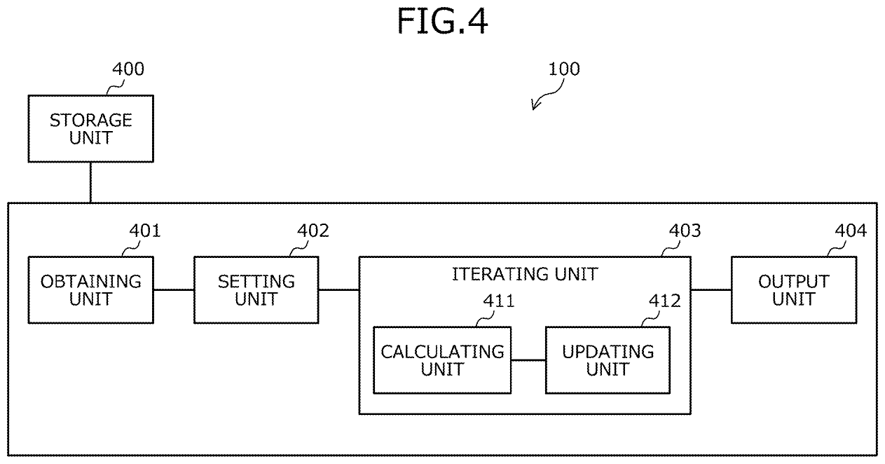

Next, an example of a functional configuration of the information processing device 100 will be described with reference to FIG. 4.

FIG. 4 is a block diagram depicting an example of a functional configuration of the information processing device 100. The information processing device 100 includes a storage unit 400, an obtaining unit 401, a setting unit 402, an iterating unit 403, and an output unit 404. The iterating unit 403 includes a calculating unit 411 and an updating unit 412.

The storage unit 400 is realized by, for example, a storage area such as the memory 302 or the recording medium 305 depicted in FIG. 3. Hereinafter, while a case in which the storage unit 400 is included in the information processing device 100 will be described, the present disclosure is not limited hereto. For example, the storage unit 400 may be included in a device different from the information processing device 100, and the storage content of the storage unit 400 may be referable from the information processing device 100.

The obtaining unit 401 to the output unit 404 function as an example of a controller. Specifically, the functions of the obtaining unit 401 to the output unit 404 are realized, for example, by causing the CPU 301 to execute a program stored in a storage area such as the memory 302 or the recording medium 305 depicted in FIG. 3 or by the network I/F 303. The process results of the functional units are stored to, for example, a storage area such as the memory 302 or the recording medium 305 depicted in FIG. 3.

The storage unit 400 stores various types of information referred to or updated in the processes of the functional units. The storage unit 400 stores, for example, structural information indicating the structure of a molecule of interest. A molecule of interest includes multiple atoms. The structural information includes, for example, coordinates of each atom among the multiple atoms forming the molecule of interest. The structural information includes, for example, the type of each of the multiple atoms forming the molecule of interest. The structure information is obtained by, for example, the obtaining unit 401.

The storage unit 400 stores, for example, fragment information indicating each of multiple fragments obtained by dividing the structure of the molecule of interest. The fragment information includes, for example, coordinates of each one or more atoms belonging to the fragment. The fragment information includes, for example, the type of each of the one or more atoms belonging to the fragment. Fragments share, for example, at least one atom. The fragment information is set by the setting unit 402, for example. The fragment information may be obtained by the obtaining unit 401, for example.

The storage unit 400 stores, for example, the first optimization calculation method. The first optimization calculation method is, for example, CCSD, FCI, VQE, or the like. The first optimization calculation method is set in advance by a user, for example. The first optimization calculation method may be obtained by the obtaining unit 401, for example.

The storage unit 400 stores, for example, the second optimization calculation method having noise resistance. The second optimization calculation method is, for example, a Newton method having noise resistance. The second optimization calculation method is set in advance by the user, for example. The second optimization calculation method may be obtained by the obtaining unit 401, for example.

The obtaining unit 401 obtains various types of information used for the processes of the functional units. The obtaining unit 401 stores the obtained various types of information to the storage unit 400 or outputs the obtained various types of information to the functional units. In addition, the obtaining unit 401 may output various types of information stored in the storage unit 400 to the functional units. The obtaining unit 401 obtains various types of information based on, for example, an operation input of a user. For example, the obtaining unit 401 may receive various types of information from a device different from the information processing device 100.

The obtaining unit 401 obtains, for example, a processing request requesting execution of quantum chemical calculation for a molecule of interest. The processing request may include, for example, structure information. The processing request may include, for example, fragment information. Specifically, the obtaining unit 401 obtains the processing request by receiving an input of the processing request. Specifically, the obtaining unit 401 may obtain the processing request by receiving the processing request from another computer. The other computer is, for example, the client device 202.

The obtaining unit 401 obtains, for example, structure information. Specifically, the obtaining unit 401 obtains the structure information by receiving an input of the structure information. Specifically, the obtaining unit 401 may obtain the structure information by receiving the structure information from another computer. The other computer is, for example, the client device 202. Specifically, the obtaining unit 401 may obtain the structure information by extracting the structure information from the processing request.

The obtaining unit 401 obtains fragment information, for example. Specifically, the obtaining unit 401 obtains the fragment information by receiving an input of the fragment information. Specifically, the obtaining unit 401 may obtain the fragment information by receiving the fragment information from another computer. The other computer is, for example, the client device 202. Specifically, the obtaining unit 401 may obtain the fragment information by extracting the fragment information from the processing request.

The obtaining unit 401 obtains, for example, the first optimization calculation method. Specifically, the obtaining unit 401 obtains the first optimization calculation method by receiving an input of the first optimization calculation method. Specifically, the obtaining unit 401 may obtain the first optimization calculation method by receiving the first optimization calculation method from another computer. The other computer is, for example, the client device 202.

The obtaining unit 401 obtains, for example, the second optimization calculation method. Specifically, the obtaining unit 401 obtains the second optimization calculation method by receiving an input of the second optimization calculation method. Specifically, the obtaining unit 401 may obtain the second optimization calculation method by receiving the second optimization calculation method from another computer. The other computer is, for example, the client device 202.

The obtaining unit 401 may receive a start trigger for starting the process of any of the functional units. The start trigger is, for example, a predetermined operation input by the user. The start trigger may be, for example, reception of predetermined information from another computer. The start trigger may be, for example, output of predetermined information by any of the functional units. Specifically, the obtaining unit 401 regards obtaining a processing request as a start trigger for starting the process of the iterating unit 403.

The setting unit 402 sets an initial value of the Lagrange multiplier. The initial value may be set in advance by the user, for example. Thus, the setting unit 402 may calculate the density matrix corresponding to each fragment.

The setting unit 402 sets fragment information indicating each of multiple fragments obtained by dividing the structure of the molecule of interest. Fragments share, for example, at least one atom. For example, the setting unit 402 divides the structure of the molecule of interest into multiple fragments and sets fragment information indicating each fragment.

Specifically, the setting unit 402 divides the structure of the molecule of interest into multiple fragments so that each fragment includes an atom other than hydrogen and a hydrogen atom directly connected to the atom other than hydrogen. Here, specifically, the setting unit 402 may divide the structure of the molecule of interest into multiple fragments so that fragments among the resulting multiple fragments share at least one or more atoms forming the molecule. Thus, the setting unit 402 may reduce the workload on the user when setting the fragment information.

The iterating unit 403 repeatedly performs a series of processes by the calculating unit 411 and the updating unit 412 until a predetermined exit condition is satisfied. The predetermined exit condition is set in advance by the user, for example. The predetermined exit condition is, for example, that the density matrices corresponding to the respective fragments match or are similar. Accordingly, the iterating unit 403 may optimize the density matrix and the Lagrange multiplier.

The calculating unit 411 calculates a density matrix corresponding to each fragment, based on the Lagrange multiplier by using the first optimization calculation method. For example, the calculating unit 411 obtains a wave function corresponding to each fragment by solving an unconstrained eigenvalue problem based on the Lagrange multiplier and calculates a density matrix corresponding to the fragment. Thus, the calculating unit 411 may optimize the density matrix corresponding to each fragment.

When the predetermined exit condition is not satisfied, the updating unit 412 updates the Lagrange multiplier so as to maintain consistency between the fragments, based on the density matrix calculated by the calculating unit 411 using the second optimization calculation method having noise resistance. Maintaining consistency means that the density matrices match or are similar for overlap between the fragments. So as to maintain consistency between the fragments, the updating unit 412 updates the Lagrange multiplier according to the Newton-Krylov method based on the density matrix by using, for example, the second optimization calculation method.

Specifically, the updating unit 412 uses the second optimization calculation method and according to the Newton-Krylov method, updates the Lagrange multiplier in a direction in which the calculated density matrices match with each other for at least one or more atoms shared by the fragments. Accordingly, the updating unit 412 may optimize the Lagrange multiplier. The updating unit 412 may suppress the adverse effect of noise on the optimization calculation for updating the Lagrange multiplier and may improve the accuracy of updating the Lagrange multiplier. Thus, the updating unit 412 may improve the accuracy of calculating the energy of the molecule using the molecule dividing method.

When the predetermined exit condition is satisfied, the iterating unit 403 calculates the energy of the molecule based on the most recently updated Lagrange multiplier and the most recently calculated density matrix. Thus, the iterating unit 403 may accurately calculate the energy of the molecule based on the optimized Lagrange multiplier and the density matrix and may complete the quantum chemical calculation.

The output unit 404 outputs a processing result of at least one of the functional units. The output format is, for example, display on a display, print output to a printer, transmission to an external device by the network I/F 303, or storage to a storage area such as the memory 302 or the recording medium 305. Thus, the output unit 404 may notify the user of the processing result of at least one of the functional units, and the convenience of the information processing device 100 may be improved.

For example, when the predetermined exit condition is satisfied, the output unit 404 outputs the Lagrange multiplier updated most recently and the density matrix calculated most recently. Specifically, the output unit 404 outputs the Lagrange multiplier updated most recently and the density matrix calculated most recently so that the user may refer to each. Specifically, the output unit 404 may transmit the Lagrange multiplier updated most recently and the density matrix calculated most recently to another computer. The other computer is, for example, the chemical calculating device 201 or the client device 202. As a result, the output unit 404 may enable the optimized Lagrange multiplier and the density matrix to be available externally. The output 404 may enable the energy of the molecule of interest to be calculated externally.

For example, when the predetermined exit condition is satisfied, the output unit 404 outputs the energy of the molecule of interest calculated by the iterating unit 403 as a result of performing the quantum chemical calculation for the molecule of interest. Specifically, the output unit 404 outputs the energy of the molecule of interest so that the user may refer to the energy. Specifically, the output unit 404 may transmit the value of the energy of the molecule of interest to another computer. The other computer is, for example, the client device 202. Thus, the output unit 404 may make the value of the energy of the molecule of interest available externally.

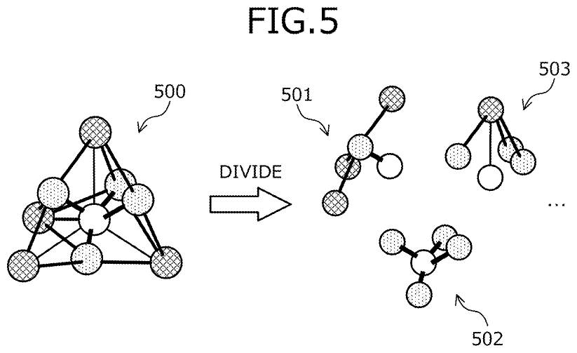

Next, an example of operation of the information processing device 100 will be described with reference to FIG. 5.

FIG. 5 is an explanatory diagram depicting an example of operation of the information processing device 100. In FIG. 5, the information processing device 100 divides the structure of a molecule of interest into multiple fragments and performs quantum chemical calculation for each fragment, thereby performing quantum chemical calculation for the entire molecule of interest and calculating the energy of the molecule of interest.

In the example depicted in FIG. 5, it is assumed that the information processing device 100 divides a structure 500 of a molecule of interest into fragments 501 to 503 and the like. When performing quantum chemical calculation for a molecule of interest, the information processing device 100 repeatedly performs a first optimization calculation process of calculating a particle density matrix and a second optimization calculation process of maintaining consistency between fragments until a predetermined exit condition is satisfied.

In the following description, the particle density matrix may be referred to as “PDM (Particle Density Matrix)”. In the following description, the first optimization calculation process may be referred to as “PDM calculation”. In the following description, the second optimization calculation process may be referred to as “matching calculation”.

In the PDM calculation, for example, a wave function ψ_{A} corresponding to each fragment A is obtained. The wave function ω_{A} is defined by the following formula (1), for example. The constraint on the following formula (1) is defined by, for example, the following formulae (2) to (4).

$${Ψ}_{A} = arg {min}_{{Ψ}_{A}} {〈 {\hat{H}}_{e m b}^{A} 〉}_{A} \qquad ( 1 )$$

$$〈 {Ψ}_{A} \vert {Ψ}_{A} 〉 = 1 \qquad ( 2 )$$

$${〈 {a}_{p}^{†} {a}_{q} 〉}_{A} = {P}_{p q}^{B} \qquad ( 3 )$$

$$∀ p , q ∈ {ℂ}_{B} ⋂ {𝔼}_{A} \qquad ( 4 )$$

Where, B is another fragment that overlaps with fragment A. Fragment B shares at least one atom with fragment A, for example. H_{emb}^{A} with {circumflex A over H} above is the Hamiltonian of fragment A. P^{B} is one PDM of fragment B. aₚ† and a_{q} are a generation operator and an annihilation operator, respectively. C_{B} and Eₐ are the central site of the fragment B and the edge site of the fragment A, respectively. < . . . >_{AS}: =<ψ_{A}| . . . |ψ_{A}>.

Formulae (1) to (4) are constrained optimization problems. Therefore, the above formulae (1) to (4) may be reduced to an unconstrained eigenvalue problem based on the Lagrange multiplier λ. The Lagrange multiplier λ is defined by the following formula (5). The unconstrained eigenvalue problem is defined by the following formulae (6) and (7).

$$λ = {( {λ}_{p , q}^{A} )}_{p , q ∈ {C}_{A} ⋂ {E}_{B} , A \neq B} \qquad ( 5 )$$

$$( {\hat{H}}_{e m b}^{A} + {\hat{λ}}_{A} ) \vert {Ψ}_{A} 〉 = {ℰ}_{A} \vert {Ψ}_{A} 〉 \qquad ( 6 )$$

$${\hat{λ}}_{A} = {\sum }_{B \neq A} [ {\sum }_{p , q ∈ {ℂ}_{B} ⋂ {𝔼}_{A}} {λ}_{p q}^{B} {a}_{p}^{†} {a}_{q} ] + μ {\sum }_{p ∈ {ℂ}_{A}} {a}_{p}^{†} {a}_{p} \qquad ( 7 )$$

By solving the eigenvalue problem for the set Lagrange multiplier, the wave function ψ_{A} corresponding to each fragment A may be obtained, and PDMP_{A} may be calculated. For example, PDMP_{A} may be calculated based on the solution of the above formula (6) in the case of λ_{A}=0 with {circumflex \hat{ } over H} above. Therefore, the PDM calculation is realized by solving the eigenvalue problem for the set Lagrange multiplier. The eigenvalue problem may be solved by, for example, an optimization calculation method. Specifically, the eigenvalue problem may be solved by the first optimization calculation method with relatively high accuracy such as CCSD, FCI, or VQE.

In the matching calculation, the Lagrange multiplier (λ_{pq}^{A}) is optimized so that ε_{BE} becomes 0, and the vector-valued function F is solved. Here, ε_{BE} is defined by the following formula (8). The vector-valued function F is defined by the following formula (9). The Lagrange multiplier may be updated by solving the vector-valued function F(λ)=0. The vector-valued function F(λ) is R^{d}→R^{d}. The vector-valued function F is dependent on the optimization calculation method.

$${ε}_{B E} = {[ \frac{1}{{N}_{c o n s}} {\sum }_{A} {\sum }_{B \neq A} {\sum }_{p , q ∈ {ℂ}_{A} ⋂ {𝔼}_{B}} {( {P}_{p q}^{A} - {P}_{p q}^{B} )}^{2} ]}^{1 / 2} \qquad ( 8 )$$

$$F : λ = {( {λ}_{p , q}^{A} )}_{p , q ∈ {C}_{A} ⋂ {E}_{B} , A \neq B} ↦ {( {P}_{p , q}^{A} ( λ ) - {P}_{p , q}^{B} ( λ ) )}_{p , q ∈ {C}_{A} ⋂ {E}_{B} , A \neq B} \qquad ( 9 )$$

In a related art, there is a method of performing matching calculation by the Newton method. In this method, specifically, the provisional solution u^{k} is updated by an update rule defined by the following formulae (10) and (11). Where, k=0, 1, . . . u^{k} corresponds to λ. J represents the Jacobian of the vector-valued function F. As the number of dimensions of u increases, when the Jacobian is calculated by a finite difference method, differences are calculated in all directions of u, and there is a problem in that an increase in the amount of processing necessary when matching calculation is performed is caused.

J(u^{k})δu^{k}=−F(u^{k}) (10)

u^{k+1}=u^{k}+δu^{k} (11)

There is also a method of performing matching calculation by the Newton-Krylov method. In this method, specifically, an approximate expression of a Jacobian defined by the following formula (12) is calculated. Thus, Jv may be calculated for a specific direction vector v for each iteration of the Newton-Krylov method. Here, when the vector-valued function F is calculated, the first optimization calculation method with relatively high accuracy such as CCSD, FCI, or VQE is performed.

Jν≈[F(u+ϵv)−F(u)]/ϵ (12)

However, in the various methods described above, the vector-valued function F, the provisional solution u^{k}, or the like may be affected by noise. Therefore, the information processing device 100 performs the matching calculation by an update rule of the Newton method having noise resistance. The update rule of the Newton method having noise resistance is defined by, for example, the following formula (13). The u^{k} with {circumflex \hat{ }} above is the provisional solution at the k-th iteration of the algorithm. The u^{k} with {circumflex \hat{ }} above corresponds to λ. Dₖ is a regular matrix for suppressing the adverse effect of noise. Jₖ† is a pseudo-inverse matrix. The pseudo-inverse matrix may be calculated by the Newton-Krylov method.

$${{D}_{k}^{- 1} \}}_{k} ( {\hat{u}}^{k} ) δ {u}^{k} = - \hat{F} ( {\hat{u}}^{k} ) \to {\hat{u}}^{k + 1} ← {D}_{k} {J}_{k}^{†} ( {\hat{u}}^{k} ) \hat{F} ( {\hat{u}}^{k} ) \qquad ( 13 )$$

The information processing device 100 calculates the Jacobian of the vector-valued function F by an approximation formula of the Jacobian defined by the above formula (12). Accordingly, the information processing device 100 may suppress an adverse effect of noise on the second optimization calculation process that maintains consistency between fragments. Thus, the information processing device 100 may improve the accuracy of calculating the energy of the molecule using BE.

Here, while a case in which the first optimization calculation method with relatively high accuracy such as CCSD, FCI, or VQE is performed when calculating the vector-valued function F has been described, the present disclosure is not limited hereto. For example, the Jacobian may be approximated by calculating a function that substitutes for the vector-valued function F using the second optimization calculation method such as a Moller-Plesset method that tends to have a smaller calculation amount than CCSD, FCI, VQE, or the like.

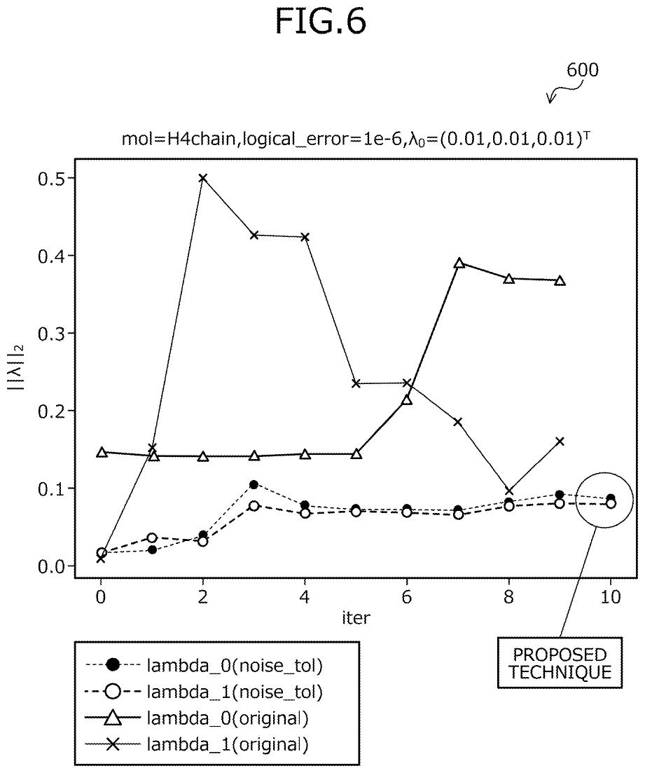

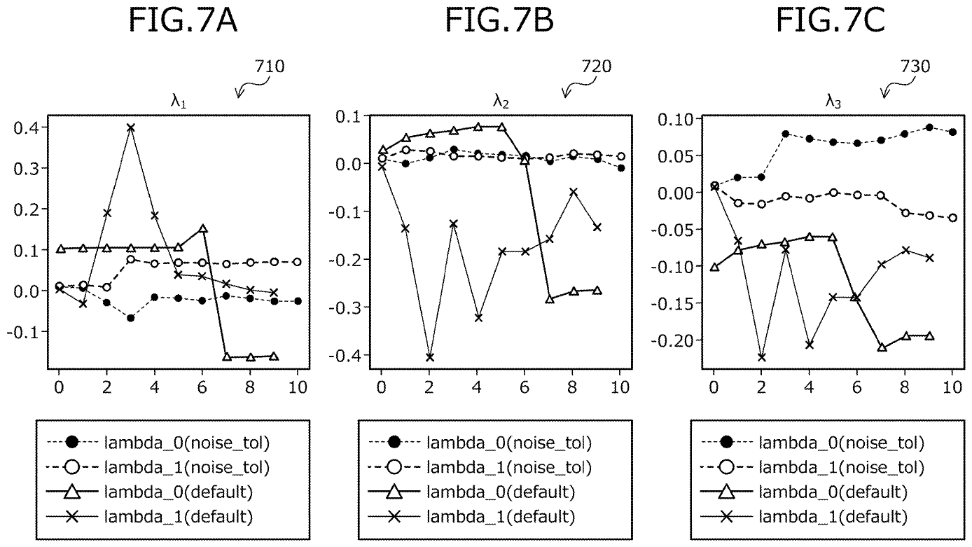

Next, an example of an effect of the information processing device 100 will be described with reference to FIGS. 6, 7A, 7B, and 7C.

FIGS. 6 and 7 are explanatory diagrams depicting an example of an effect of the information processing device 100. In FIGS. 6 and 7, it is assumed that the information processing device 100 calculates the energy of a molecule H4 chain using the BE to which the Newton method having noise resistance is applied by the quantum simulator with noise. In addition, it is assumed that the energy of the molecule H4 chain is calculated using BE to which the Newton's method having no noise resistance is applied by the conventional method.

In FIG. 6, a graph 600 depicts a change in the norm ∥λ∥₂ of λ with respect to a change in the iteration when the information processing device 100 calculates the energy of the molecule H4 chain. The graph 600 depicts a change in the norm ∥λ∥₂ of λ with respect to a change in the iteration when the energy of the molecule H4 chain is calculated by the conventional method. Specifically, the identifier noise_tol corresponds to the information processing device 100. The “original” identifier corresponds to the conventional method. A horizontal axis of the graph 600 represents an iteration. A vertical axis of the graph 600 represents the norm ∥λ∥₂. As depicted in the graph 600, the information processing device 100 may stabilize the behavior of the norm ∥λ∥₂ and suppress the adverse effect of noise as compared to the conventional method. Next, FIGS. 7A, 7B, and 7C will be described. FIGS. 7A 7B and 7C are each an explanatory diagram depicting an example of an effect of the information processing device 100.

In FIG. 7A, a graph 710 depicts a change in the element λ₁ of λ with respect to a change in the iteration when the information processing device 100 calculates the energy of the molecule H4 chain. The graph 710 depicts a change in the element λ₁ of λ with respect to a change in the iteration when the energy of the molecule H4 chain is calculated by the conventional method. Specifically, the identifier noise_tol corresponds to the information processing device 100. The “original” identifier corresponds to the conventional method. A horizontal axis of the graph 710 represents an iteration. A vertical axis of the graph 710 represents the element λ₁.

In FIG. 7B, a graph 720 depicts a change in the element λ₂ of λ with respect to a change in the iteration when the information processing device 100 calculates the energy of the molecule H4 chain. The graph 720 depicts the change of the element λ₂ of λ with respect to the change of the iteration in a case where the energy of the molecule H4 chain is calculated by the conventional method. Specifically, the identifier of noise_tol corresponds to the information processing device 100. The “original” identifier corresponds to the conventional method. A horizontal axis of the graph 720 represents an iteration. A vertical axis of the graph 720 represents the element λ₂.

In FIG. 7C, a graph 730 depicts a change in the element λ₃ of λ with respect to a change in the iteration when the information processing device 100 calculates the energy of the molecule H4 chain. The graph 730 depicts the change of the element λ₃ of λ with respect to the change of the iteration in a case where the energy of the molecule H4 chain is calculated by the conventional method. Specifically, the identifier noise_tol corresponds to the information processing device 100. The “original” identifier corresponds to the conventional method. A horizontal axis of the graph 730 represents an iteration. A vertical axis of the graph 730 represents the element λ₃.

As depicted in the graphs 710, 720, and 730, the information processing device 100 may stabilize the behaviors of the element λ₁, the element λ₂, and the element λ₃ and suppress the adverse effect of noise as compared with the conventional method. As described above, the information processing device 100 may improve the accuracy of calculating the energy of the molecule as compared with the conventional method.

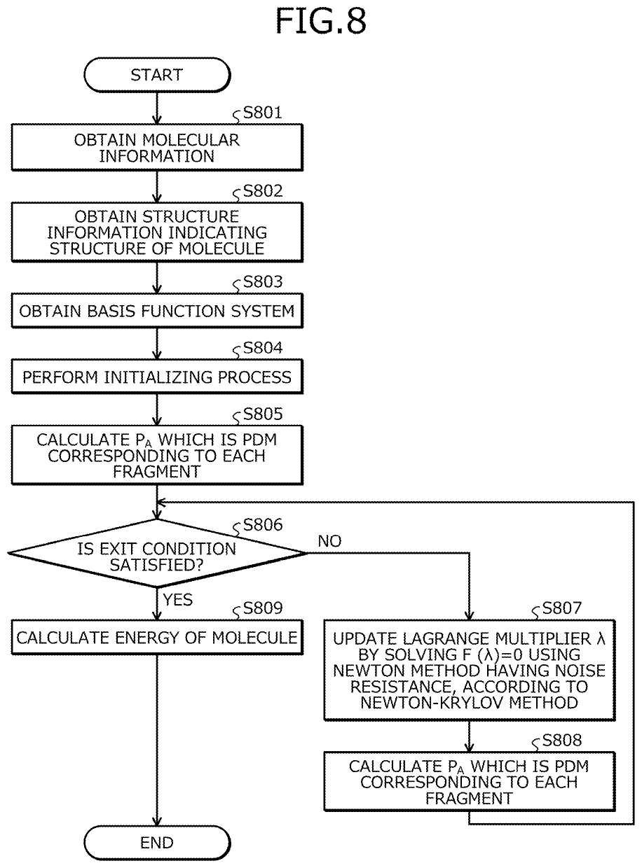

Next, an example of a procedure of an overall process executed by the information processing device 100 will be described with reference to FIG. 8. The overall process is implemented by, for example, the CPU 301, storage areas such as the memory 302 and the recording medium 305, and the network I/F 303 depicted in FIG. 3.

FIG. 8 is a flowchart depicting an example of a procedure of the overall process. In FIG. 8, the information processing device 100 obtains molecular information (step S801). Next, the information processing device 100 obtains structure information indicating the structure of the molecule (step S802). Then, the information processing device 100 obtains a basis function system (step S803).

Next, the information processing device 100 performs an initializing process, sets the Lagrange multiplier λ=0, and sets the upper limit of the number of iterations (step S804). Then, based on the Lagrange multiplier λ, the information processing device 100 calculates P_{A} which is PDM corresponding to each fragment (step S805).

Next, the information processing device 100 determines whether an exit condition is satisfied (step S806). Here, when the exit condition is not satisfied (step S806: NO), the information processing device 100 proceeds to the process at step S807. On the other hand, when the exit condition is satisfied (step S806: YES), the information processing device 100 proceeds to the process at step S809.

At step S807, the information processing device 100 updates the Lagrange multiplier λ by solving F(λ)=0 using the Newton method having noise resistance, according to the Newton-Krylov method (step S807).

Next, based on the Lagrange multiplier λ, the information processing device 100 calculates P_{A} which is PDM corresponding to each fragment (step S808). Then, the information processing device 100 returns to the process at step S806.

At step S809, the information processing device 100 calculates the energy of the molecule based on the Lagrange multiplier λ and (P_{A})_{A} corresponding to each fragment (step S809). Then, the information processing device 100 ends the entire process. Thus, the information processing device 100 may improve the accuracy of calculating the energy of the molecule while reducing the amount of processing necessary to calculate the energy of the molecule.

Here, the information processing device 100 may change the order of the processes of some steps in FIG. 8. For example, the order of the processes at steps S801 to S804 may be changed. In addition, the information processing device 100 may omit the processes of some steps in FIG. 8. For example, the process at step S610 may be omitted.

The information processing device 100 may be applied to fields such as drug discovery and material development, for example. Specifically, in the field of drug discovery, material development, or the like, the information processing device 100 may be applied to a case where it is desired to perform quantum chemical calculation for calculating the energy of a molecule in order to analyze the structure or properties of the molecule that is a candidate for a drug or a material. As a result, the information processing device 100 may maintain the accuracy of the quantum chemical calculation while reducing the amount of processing necessary to perform the quantum chemical calculation, may easily calculate the energy of the molecule, and may contribute to the fields of drug discovery, material development, and the like.

In the above description, while a case in which the information processing device updates the Lagrange multiplier has been focused on for simplification of description, the present disclosure is not limited hereto. For example, the information processing device may perform optimization calculation for updating p of the chemical potential. At this time, the information processing device updates p of the chemical potential so that the total number of electrons of each fragment matches the number of electrons of the molecule, thereby maintaining the consistency between the fragments.

As described above, according to the information processing device 100, a density matrix corresponding to each of multiple fragments obtained by dividing a structure of a molecule may be calculated based on a Lagrange multiplier. According to the information processing device 100, it is possible to update the Lagrange multiplier so as to maintain the consistency between the fragments according to the Newton-Krylov method using the optimization calculation method having noise resistance, based on the calculated density matrices. Thus, the information processing device 100 may improve the accuracy of calculating the energy of the molecule.

According to the information processing device 100, when the predetermined exit condition is satisfied, the energy of the molecule may be calculated based on the Lagrange multiplier updated most recently and the density matrix calculated most recently. Thus, the information processing device 100 may complete the quantum chemical calculation.

According to the information processing device 100, it is possible to specify multiple fragments obtained by dividing the structure of a molecule such that the fragments share at least one or more atoms forming the molecule. According to the information processing device 100, it is possible to update the Lagrange multiplier in a direction in which the calculated density matrices match each other with respect to at least one or more atoms shared by fragments. Accordingly, the information processing device 100 may easily update the Lagrange multiplier with high accuracy.

According to the information processing device 100, the calculated value of the energy of the molecule may be output. Thus, the information processing device 100 may enable external reference of the result of performing the quantum chemical calculation.

The information processing method described in the present embodiment may be implemented by executing a prepared program on a computer such as a personal computer and a workstation. The program is stored on a non-transitory, computer-readable recording medium such as a hard disk, a flexible disk, a compact disk read-only memory (CD-ROM), a magneto-optical (MO) disk, and a digital versatile disc (DVD), read out from the computer-readable medium, and executed by the computer. The program may be distributed through a network such as the Internet.

According to the information processing device 100, it is possible to improve the accuracy of calculating the energy of the molecule using the molecule dividing method.

All examples and conditional language provided herein are intended for pedagogical purposes of aiding the reader in understanding the invention and the concepts contributed by the inventor to further the art, and are not to be construed as limitations to such specifically recited examples and conditions, nor does the organization of such examples in the specification relate to a showing of the superiority and inferiority of the invention. Although one or more embodiments of the present invention have been described in detail, it should be understood that the various changes, substitutions, and alterations could be made hereto without departing from the spirit and scope of the invention.

What is claimed is:

1. A computer-readable recording medium storing therein a program for causing a computer to execute a process, the process comprising:
calculating density matrices respectively corresponding to a plurality of fragments obtained by dividing a structure of a molecule based on a Lagrange multiplier; and
updating the Lagrangian multiplier based on the calculated density matrices when a predetermined exit condition is not satisfied, the Lagrangian multiplier being updated using a noise-resistant optimization calculation method according to a Newton-Krylov method so as to maintain consistency among the plurality of fragments, wherein
the process is repeatedly executed until the predetermined exit condition is satisfied.

2. The computer-readable recording medium according to claim 1, the process further comprising calculating an energy of the molecule based on the Lagrange multiplier updated most recently and the density matrix calculated most recently, when the predetermined exit condition is satisfied.

3. The computer-readable recording medium according to claim 1, wherein
fragments of the plurality of fragments share at least one or more atoms forming the molecule, and
the updating includes, when the predetermined exit condition is not satisfied, updating the Lagrange multiplier in a direction so that the calculated density matrices match each other with respect to the at least one or more atoms shared by the fragments of the plurality of fragments.

4. The computer-readable recording medium according to claim 2, wherein the calculated energy of the molecule is output.

5. An information processing method, comprising:
calculating density matrices respectively corresponding to a plurality of fragments obtained by dividing a structure of a molecule based on a Lagrange multiplier; and
updating the Lagrangian multiplier based on the calculated density matrices when a predetermined exit condition is not satisfied, the Lagrangian multiplier being updated using a noise-resistant optimization calculation method according to a Newton-Krylov method so as to maintain consistency among the plurality of fragments, wherein
the process is repeatedly executed until the predetermined exit condition is satisfied.

6. An information processing device comprising:
a memory; and
a processor coupled to the memory, the processor configured to:
calculate density matrices respectively corresponding to a plurality of fragments obtained by dividing a structure of a molecule based on a Lagrange multiplier; and
update the Lagrangian multiplier based on the calculated density matrices when a predetermined exit condition is not satisfied, the Lagrangian multiplier being updated using a noise-resistant optimization calculation method according to a Newton-Krylov method so as to maintain consistency among the plurality of fragments, wherein
the process is repeatedly executed until the predetermined exit condition is satisfied.
